(19) United States
(12) Patent Application Publication
(10) Pub. No.: US 2021/0031044 A1
(43) Pub. Date: Feb. 4, 2021

(54) COGNITIVE AND MEMORY ENHANCEMENT SYSTEMS AND METHODS

(71) Applicant: Mayo Foundation for Medical Education and Research, Rochester, MN (US)

(72) Inventors: Michal T. Kucewicz, Rochester, MN (US); Brent M. Berry, Rochester, MN (US); Gregory A. Worrell, Rochester, MN (US); Vaclav Kremen, Rochester, MN (US)

(21) Appl. No.: 16/981,823

(22) PCT Filed: Mar. 19, 2019
(86) PCT No.: PCT/US2019/022904

§ 371 Date: Sep. 17, 2020

Related U.S. Application Data
(60) Provisional application No. 62/645,257, filed on Mar. 20, 2018.

(51) Int. Cl. A61N 1/36; A61N 1/04; A61N 1/05

(52) U.S. Cl. CPC A61N 1/36092; A61N 1/0531; A61N 1/36135; A61N 1/0456

(57) ABSTRACT

Electrical stimulation of the brain in the lateral temporal cortex has been discovered to enhance memory performance. Also, consistent patterns of pupil response have been discovered to exist across and within distinct phases during encoding and recall of word lists and it is known that these pupillary changes also correlate with intracranial electrophysiologic activity. This document also describes systems and methods for enhancing memory and/or cognitive performance using these features as input for the delivery of electrical stimulation to the lateral temporal cortex of the brain.

### CROSS-REFERENCE TO RELATED APPLICATIONS

This application claims priority to U.S. Application Ser. No. 62/645,257, filed on Mar. 20, 2018. The disclosure of the prior application is considered part of the disclosure of this application, and is incorporated in its entirety into this application.

### BACKGROUND

### 1. Technical Field

This document relates to systems and methods for enhancing cognition, with specific applications to memory performance. For example, this document relates to systems and methods that enhance performance of any cognitive function, and with specific application to memory performance by delivering electrical stimulation to the lateral temporal cortex of the brain with or without a system that detects change in a patient's eye, such as pupil dilation, to keep the brain in an optimal cognitive function state.

### 2. Background Information

Deficits in memory and cognition present a major therapeutic challenge in a wide spectrum of brain disorders. In addition, there are multiple applications where optimizing cognitive function would be beneficial. There is a need for new approaches to cognitive enhancement that would target individualized therapy directed at specific brain regions and thus overcome limitations of current pharmacological and behavioral therapies. Electrical stimulation of discrete areas in the brain has been applied to a range of neurological and neuropsychiatric disorders without a clear understanding of how it modulates electrophysiological activities, and little is known specifically about the effect of direct electrical stimulation of the brain on memory. Recent studies have reported mixed effects using various approaches to stimulation in mesial temporal lobe structures, including the hippocampus, entorhinal cortex, and fornix (Direct Electrical Stimulation of the Human Entorhinal Region and Hippocampus Impairs Memory; Jacobs J, Miller J, Lee S A, Coffey T, Watrous A J, Sperling M R, Sharan A, Worrell G, Berry B, Lega B, Jobst B C, Davis K, Gross R E, Sheth S A, Ezzyat Y, Das S R, Stein J, Gorniak R, Kahana M J, Rizzuto D S: Neuron. 2016 Dec. 7; 92(5):983-990. doi: 10.1016/j.neuron.2016.10.062). Prior studies investigated different memory functions using a variety of spatial and non-spatial tasks in patient population presenting a range of cognitive performances.

Pupil size has been associated with cognitive processes underlying perception, attention and action for external stimuli. Pupil dilation and constriction has been shown to indicate interest in the content of the presented visual stimuli. It is also known to indicate general mental activity and correlate with task difficulty. Pupil size is also shown to correlate with neuro-electrophysiologic activity such as high frequency oscillations (aNeuron; 2015 Jul. 1; 87(1):179-92. doi:10.1016/j.neuron.2015.05.038. Epub 2015 Jun. 11).

Recent studies have shown that high-resolution tracking of pupil size alone or together with other modalities (brain electrophysiology, sympathetic nervous activity tracking) can be used to predict perception of specific stimuli and even the voluntary decisions about attending the stimuli.

### SUMMARY

This document describes that electrical stimulation of the brain in the temporal cortex has been discovered to enhance memory performance. The document also describes that consistent patterns of pupil response have been discovered to exist across and within distinct phases during encoding and recall of word lists. Further, this document describes systems and methods for enhancing memory performance by using the detection of eye changes as a trigger for the delivery of electrical stimulation to the lateral temporal cortex of the brain.

In one aspect, this disclosure is directed to a system for cognitive performance or memory enhancement therapy. The system, includes a controller; an eye-change detection sub-system in signal communication with the controller; and an electrical brain stimulation sub-system in signal communication with the controller.

Such a system may optionally include one or more of the following features. The eye-change detection sub-system may comprise one or more cameras. The controller may be configured for adaptive training. The system may be a hand-held device.

In another aspect, this disclosure is directed to a method for enhancing memory or cognitive performance of a patient. The method includes detecting a change in an eye of the patient; comparing the change to predetermined criteria; and in response to the change meeting the predetermined criteria, delivering electrical brain stimulation.

Such a method for enhancing memory or cognitive performance of a patient may optionally include one or more of the following features. The change may comprise a dilation or constriction of a pupil. The change may comprise an eye movement or a change in a gaze of the eye of the patient. The electrical brain stimulation may be delivered to a lateral temporal cortex of the patient.

In another aspect, this disclosure is directed to a method for enhancing memory or cognitive performance of a patient. The method includes detecting a change in an eye of the patient; correlating the detected change in the eye of the patient with electrophysiologic signals from within a brain of the patient; and in response to the correlation meeting predetermined criteria, delivering electrical brain stimulation.

Particular embodiments of the subject matter described in this document can be implemented to realize one or more of the following advantages. In some embodiments, memory performance can be enhanced in an effective and efficient manner. To achieve such results, the timing of the delivery of electrical stimulation to the lateral temporal cortex of the brain can be optimized in a closed-loop sense by using pupil response also or with other modalities of data, but can also be achieved in an open loop fashion with direct stimulation to the lateral temporal cortex.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described herein. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description herein. Other features, objects, and advantages of the invention will be apparent from the description and drawings, and from the claims.

### DESCRIPTION OF THE DRAWINGS

FIG. 1 pertains to tests that show stimulation in the lateral temporal cortex enhances verbal memory performance. “Panel A” is a diagram of free recall verbal memory task design comprising three successive stages. “Panel B” shows a stimulation site on the lateral temporal cortex (red electrode pair) and in parahippocampal cortex (blue circle) used for subject 1111. “Panel C” shows the memory performance of subject 1111 across all stimulation sessions. Overall session scores are in bold, broken down into scores on stimulated (left side thunderbolt) and nonstimulated word lists (right side). “Panel D” shows the memory performance of all four subjects stimulated in the lateral temporal cortex and another target in two patients (*−p<0.05, permutation test). “Panel E” shows paired t-test comparison of subject memory performance on the stimulated and non-stimulated lists (**−p<0.01). All data are shown as mean±SEM.

FIG. 2 pertains to the localization of the temporal cortex stimulation sites relative to task-induced high gamma activity. “Panel a” is a diagram of an example 8×8 grid of electrodes used to stimulate temporal cortex in subject 1050 (red marks the stimulating electrode pair). “Panel b” is a surface plot displaying peak power values of high gamma activity induced by presentation of words for memory encoding interpolated across all 64 grid electrodes on the underlying brain surface of subject 1050 (electrodes are marked with blue dots). “Panel c” shows analogous surface plots are displayed for the remaining three patients (subject 1176 was stimulated from a depth electrode). The stimulation sites (in red) localize in proximity to high gamma activity foci in the lateral temporal cortex of subjects 1050 and 1111.

FIG. 3 pertains to stimulation-induced memory enhancement being specific to the lateral temporal cortex. “Panel a” shows localization of four stimulation sites in the middle temporal gyrus of the lateral temporal cortex (red), which is highlighted with white lining, and 19 other sites tested (black) visualized in a unified transparent brain surface. “Panel b” shows that stimulation enhances memory performance in the four subjects stimulated in the lateral temporal cortex (TC; red bars) as compared to the other brain areas studied (PH: parahippocampal region, HP: hippocampus, PF: prefrontal cortex). Tukey-Kramer post-hoc ANOVA comparison (right side) shows that TC means are significantly higher than PH, HP, PF (p<0.05).

FIG. 4 shows that pupil dilation is modulated by different phases of the free recall verbal memory task. “Panel a” shows trial-averaged changes in pupil size of one subject across four phases of the free recall task. Shaded areas mark epochs of word presentation on the screen and their recall with blank screen. Consistent and stereotypical pupil responses across the trials reveal gradually increasing size in successive task phases. “Panel b” shows mean changes in pupil size summarized in 12 s time bins of the four task phases for every subject (colors are different subjects). “Panel c” shows post-hoc ANOVA group comparisons of means from the task phase bins (as in “panel b”) shows that pupil area was decreased during countdown and increased during recall. The red dotted lines are 95% confidence intervals. The two phases are characterized by no cognitive load in the former and maximum load in the latter.

FIG. 5 shows that pupil size is increased in response to free recall of remembered words. “Panel a” shows an example of pupil area modulation during free recall of remembered words from one recall trial. Red lines mark the start time of word vocalization. This shows recall of words is associated with pupil dilation with no changes in the screen display. “Panel b” shows mean pupil responses from all recalled word epochs in one patient are aligned to the onset of vocalization (left). Notice the consistent dilation starting before and peaking at the time of vocalization. Mean pupil area in ±1 s epochs around the word vocalization (‘during recall’) is significantly greater than in the remaining recall epochs (‘outside recall’) with no vocalization (**−p<0.01). “Panel c” shows across-subject comparison (colors are different subjects) of the pupil area in the two epoch types shows consistently more dilated pupil during recall of remembered words (*−p<0.05).

FIG. 6 shows that remembered and forgotten words show different pupil responses during memory encoding. “Panel a” shows an example list of words presented in a sequence during encoding trials with subsequently recalled (red) and forgotten (blue) words. Mean pupil responses to presentation of words on the two trial types (right) in one example subject reveal more dilated peak response during encoding of the recalled words (horizontal bar below the asterisks indicates 50 ms bins with significant difference with p<0.01). Shaded area marks the time of word presentation on the screen. “Panel b” shows the mean memory performance of the ten subjects. “Panel c” shows subject-averaged pupil response to word encoding is presented as in “Panel a.” The pupil was more constricted on the recalled word trials just before the screen presentation, and more dilated at the peak response during encoding (bars indicate the time bins of the greatest difference). “Panel d” is a comparison of the subject means (left) and peak/trough values (right) in the epochs ‘Before’ and ‘After’ presentation onset (see “Panel c”) confirms differential modulation of the pupil size between the trials with recalled and forgotten words (**−p<0.01, *−p<0.05 with Bonferroni correction for multiple comparisons).

FIG. 7 schematically depicts a system for enhancing memory performance that uses eye changes as a trigger in a closed-loop fashion (or can be set to stimulate in an open-loop fashion) for the delivery of electrical stimulation to the lateral temporal cortex of the brain.

FIG. 8 is a flow chart depicting a method for enhancing memory performance that uses eye changes as a trigger for the delivery of electrical stimulation to the lateral temporal cortex of the brain.

Like reference numbers represent corresponding parts throughout.

### DETAILED DESCRIPTION

This document describes that electrical stimulation of the brain in the lateral temporal cortex has been discovered to enhance memory performance. The document also describes that consistent patterns of pupil response exist across and within distinct phases during encoding and recall of word lists. Further, this document describes systems and methods for enhancing memory performance via open or close loop design by using eye changes as a trigger for the delivery of electrical stimulation to the lateral temporal cortex of the brain.

### Human Memory Enhancement through Electrical Brain Stimulation in the Lateral Temporal Cortex

INTRODUCTION: The aim of this study was to compare the effect of direct brain stimulation on memory performance in four brain regions supporting declarative memory, including two regions outside of the mesial temporal lobe—dorsolateral prefrontal cortex and lateral temporal cortex. Direct electrical stimulation of the lateral temporal cortex was previously shown by Penfield and Perot (1963) to evoke multi-sensory experience of past events, but was not explored in a paradigm to assess memory enhancement. This study employed classic tasks for verbal memory performance to study the effect of stimulation on memory in individual patients and across groups of patients stimulated in the four brain regions.

SUMMARY: This study investigated the effect of stimulation in four brain regions known to support declarative memory: hippocampus, parahippocampal neocortex, prefrontal cortex and temporal cortex. Intracranial electrode recordings with stimulation were used to assess verbal memory performance in a group of 22 patients (9 males). Electrical brain stimulation in the temporal cortex (paired t-test, p=0.0067), but not in any other of the four regions involved in human declarative memory system, enhanced memory performance on a group level and in individual patients. This selective enhancement was observed both on the group level, and for two of the four individual subjects stimulated in the temporal cortex. In these studies, individual subjects performed repeated stimulation and control sessions without stimulation. All patients stimulated in the left lateral temporal cortex showed evidence for positive modulation of memory performance, with one subject even reporting a strong subjective experience of memory enhancement. This study shows that electrical stimulation in specific brain areas can enhance verbal memory performance in humans. Additionally, this study investigated high gamma band electrophysiological activity during the non-stimulation memory encoding tasks as a biomarker to map and identify potential stimulation targets similar to the previous work in animals, which presents the first of its kind in the field of human brain stimulation.

MATERIALS AND METHODS: The effect of stimulation on memory performance was investigated in epilepsy patients undergoing evaluation for resective surgery with intracranial subdural and depth electrode arrays in multiple cortical and subcortical brain regions. This study focused on 22 patients implanted in the four brain regions (Table 1, 2) of the cortical-hippocampal declarative memory system. Basic clinical information together with the epilepsy pathology and verbal memory performance is summarized in Table 1.

TABLE 1
Summary of the study participants. Patient demographic data is presented together with clinical
observations from structural magnetic resonance imaging (MRI), clinically identified seizure
onset zones (SOZ), and pathology for those subjects who underwent resective surgery.

| | | | | | | | | | Verbal |
|---|---|---|---|---|---|---|---|---|---|
| | | | | | | Brain | Language | | Memory |
| Subject No. | Age | Gender | Handedness | SOZ | MRI | Pathology | Lateralization | VIQ | Deficits |
| R1001P | 48 | F | R | right TC | Normal | Gliosis | L | 81 | None |
| R1006P | 20 | F | R | right FC | MCD | Gliosis | L | 91 | None |
| R1016M | 31 | F | R | left FC | Normal | Gliosis | — | 71 | None |
| R1018P | 47 | M | L | left FC, left | Normal | — | L | 85 | None |
| R1020J | 48 | F | L | right TC, right FC | Abnormal | Gliosis | L | 98 | Mild |
| R1022J | 24 | M | R | | Atrophy Gliosis/ | | L | 81 | None |
| | | | | | Encephalomalacia | | | | |
| R1024E | 36 | F | R | right OPC | Normal | Gliosis | L | 100 | None |
| R1026D | 24 | F | R | Left aTC, left OC | MTS, | | bilateral | 112 | None |
| | | | | | Gliosis | | | | |
| R1027J | 48 | M | R | Right TC, right IC, | Abnormal | | L | 93 | None |
| | | | | right/left | | | | | |
| R1028M | 27 | F | R | right MTL | Abnormal | CD, Gliosis | | 103 | None |
| R1029W | 33 | F | R | left FC | Abnormal | | | 108 | Mild |
| R1030J | 23 | M | L | left MTL | Normal | Gliosis | L | 106 | None |
| R1031M | 24 | M | R | Right FC, | Abnormal | | L | 110 | Moderate |
| R1033D | 31 | F | R | right TC | Atrophy | | L | 85 | None |
| R1036M | 49 | M | L | Left aTC, | MTS | HS | bilateral | 93 | Moderate |
| R1042M | 27 | F | L | right TC | MCD | | R | 114 | None |
| R1050M | 20 | M | R | left PC | Neoplasm | DNET | | 95 | Mild |
| R1060M | 36 | F | R | right TC | Normal | Gliosis | | 95 | Mild |
| R1069M | 26 | M | R | left FC | MCD | | L | | Mild |
| R1111M | 20 | M | R | left TC, left OPC, | Gliosis | Gliosis | L | 108 | None |
| | | | | left OC, | | | | | |
| R1176M | 41 | F | R | Right MTL, right IC | MTS | | L | | Moderate |
| R1177M | 23 | F | R | left TC | TS | | L | 87 | Moderate |

Abbreviations: FC—frontal cortex, TC—temporal cortex, PC—parietal cortex, OC—occipital cortex, IC—insular cortex, aTC—anterior temporal cortex, MTL—mesial temporal lobe, TPC—temporo-parietal cortex, FPC—fronto-parietal cortex, OPC—occipito-parietal cortex, CD—cortical dysplasia, HS—hippocampal sclerosis, MCD—malformation of cortical development, MTS—mesial temporal sclerosis, PMG—polymicrogyria, DNET—dysembryoplastic neuroepithelial tumor.

TABLE 2
Summary of the experiments used to assess the effect of stimulation on encoding of
word lists. Analysis was focused on 23 subject experiments that had at least two
sessions with any one stimulation target in four of the studied brain regions.

| | | | Target | Electrode | Amplitude | Frequency | Pulse width | Duration |
|---|---|---|---|---|---|---|---|---|
| Subject | Sessions | Localization | region | type | (mA) | (Hz) | (ms) | (s) |
| R1001P | 2 | left HP | HP | depth | 1.0 | 50 | 0.3 | 4.6 |
| R1006P | 2 | right HP | HP | depth | 1.0 | 50 | 0.3 | 4.6 |
| R1016M | 2 | left PF | PF | subdural | 3.5 | 50 | 0.3 | 4.6 |
| R1018P | 2 | left PF | PF | depth | 1.5 | 50 | 0.3 | 4.6 |
| R1020J | 4 | right HP | HP | depth | 1.0 | 50 | 0.3 | 4.6 |
| R1022J | 2 | left HP | HP | depth | 1.0 | 50 | 0.3 | 4.6 |
| R1024E | 3 | left HP | HP | depth | 1.0 | 50 | 0.3 | 4.6 |
| R1026D | 4 | left EC | PH | depth | 0.5 | 50 | 0.3 | 4.6 |
| R1027J | 2 | left HP | HP | depth | 1.0 | 50 | 0.3 | 4.6 |
| R1028M | 3 | right EC | PH | subdural | 1.0 | 50 | 0.3 | 4.6 |
| R1029W | 2 | left PF | PF | subdural | 3.5 | 50 | 0.3 | 4.6 |
| R1030J | 4 | left PHC | PH | depth | 0.5 | 50 | 0.3 | 4.6 |
| R1031M | 2 | right PRC | PH | depth | 1.5 | 50 | 0.3 | 4.6 |
| R1033D | 2 | left PRC | PH | depth | 1.5 | 50 | 0.3 | 4.6 |
| R1036M | 4 | left PRC | PH | depth | 1.0 | 50 | 0.3 | 4.6 |
| R1042M | 2 | right PF | PF | subdural | 1.5 | 50 | 0.3 | 4.6 |
| R1050M | 2 | left TC | TC | subdural | 1.5 | 50 | 0.3 | 4.6 |
| R1060M | 3 | right PF | PF | subdural | 3.0 | 50 | 0.3 | 4.6 |
| R1069M | 2 | left PF | PF | subdural | 2.5 | 50 | 0.3 | 4.6 |
| R1111M | 3 | left PHC | PH | depth | 0.75 | 50 | 0.3 | 4.6 |
| R1111M | 3 | left TC | TC | subdural | 1.5 | 50 | 0.3 | 4.6 |
| R1176M | 3 | left TC | TC | depth | 1.0 | 50 | 0.3 | 4.6 |
| R1177M | 4 | left TC | TC | subdural | 1.0 | 50 | 0.3 | 4.6 |

Abbreviations: PHC—parahippocampal cortex, PRC—perirhinal cortex, EC—entorhinal cortex; HP—hippocampus, TC—temporal cortex, PF—prefrontal cortex, PH—parahippocampal region.

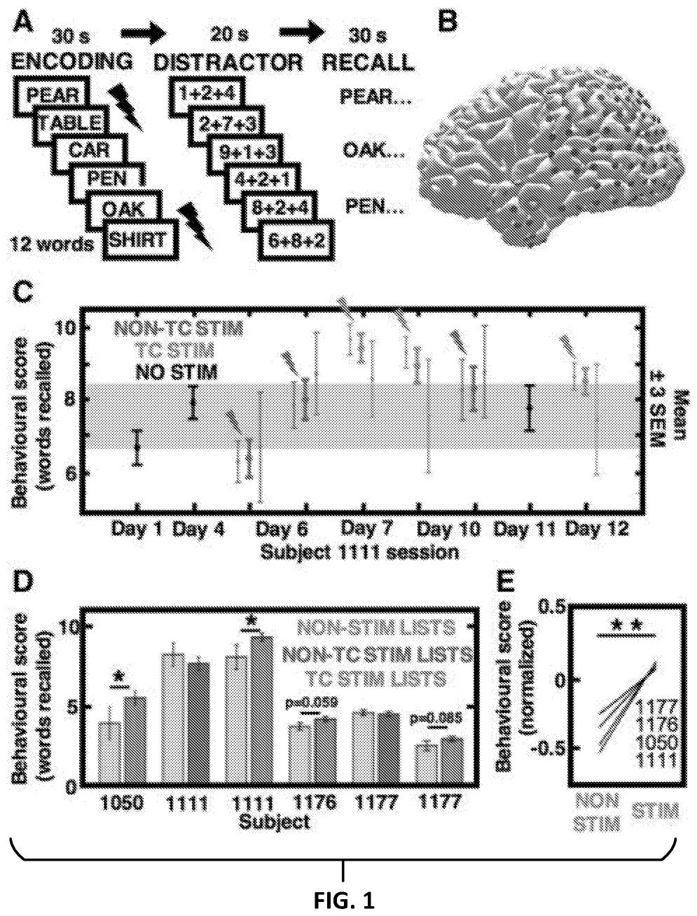

Following implantation, each patient participated in delayed free-recall memory tasks. The tasks were based on classic paradigms for probing verbal memory, in which subjects learned lists of words for subsequent recall (FIG. 1 panel A). Subjects were instructed to study lists of individual words presented sequentially on a laptop computer screen for a later memory test. Each word remained on the screen for 1600 ms, followed by a random jitter of 750-1000 ms blank interval between stimuli. Immediately following the final word in each list, participants performed a distractor task (20 seconds) consisting of a series of arithmetic problems. Following the distractor task participants were given 30 seconds to verbally recall as many words as possible from the list in any order. Each session consisted of 25 lists of this encoding-distractor-recall procedure.

Electrical stimulation was applied between pairs of adjacent electrode contacts in the specific brain regions during encoding of words for subsequent recall (FIG. 1 panel A), using a fixed set of parameters (see Table 2) taken from a recent report of memory enhancement (Suthana et al., 2012). Only the amplitude parameter was varied within a fixed narrow range with respect to other clinical factors related to safety and patient treatment. Each patient was stimulated in 1-2 brain targets and here we focused on the targets localized in the four brain regions of the declarative memory system. Specific electrodes in the target brain region were selected based on the previously described subsequent memory effect (Kahana, 2006; Sederberg et al., 2007) in the high gamma range. Safe current amplitude for stimulation was determined for the chosen electrodes in a pre-test evaluation of after-discharges. At least two stimulation sessions in one of the four brain region studied were required to be included in the data analysis (Table 2) to ensure adequate number of samples to estimate mean performance on the non-stimulated lists (n>5 lists). Additional data from single stimulation sessions were also compared as well as subset of data from stimulation of the language-dominant hemisphere. In the studied group of 22 subjects there were 7 stimulated in the parahippocampal region, 6 stimulated in the hippocampus, 4 stimulated in the temporal cortex, 6 stimulated in the prefrontal cortex, with one subject stimulated in two of these regions (Table 2). The number of sessions performed with each patient was determined by the length seizure monitoring (ranging approx. from 2-14 days) and willingness to participate in the study. The stimulation sessions were preceded by at least two record-only control sessions with no stimulation to familiarize subjects with the tasks and reduce potential learning effects. Subjects were instructed about the stimulation procedure but were blinded to the location of the stimulation site. Before starting any stimulation session the experimenter ensured that there were no after-discharges and no subjective experience of the stimulation.

All statistical tests were performed in Matlab (MathWorks Inc.) using built-in and custom written codes. The effect of stimulation on memory performance in individual subjects (FIG. 1 panel D) was assessed using a permutation test procedure—behavioral scores from all sessions with a given stimulation target were compared using difference in mean from the stimulated and non-stimulated lists, which was recalculated after randomly shuffling the list type labels 10,000 times to obtain a distribution of the shuffled difference scores. The permutation test was significant at p<0.05 level if the original difference score without label shuffling was higher (enhancement) or lower (impairment) than 95% of the shuffled distribution scores. The same permutation procedure was used to compare the mean score obtained from the patients stimulated in the temporal cortex and the other brain regions. Paired t-test was used to compare normalized mean behavioral scores on stimulated and non-stimulated lists in the four temporal cortex subjects. ANOVA test was used to compare the effect of stimulating in the four studied regions on memory performance with Tukey-Kramer post-hoc comparison of the 95% confidence intervals of the means.

RESULTS: Regarding the effect of stimulation in the lateral temporal cortex, first, the study showed that stimulation in the dominant lateral temporal neocortex of a subject with multiple stimulation sessions (FIG. 1 panel B) increased the number of remembered words above the normal range, as compared to sessions with stimulation in parahippocampal region (FIG. 1 panel C). In contrast to the parahippocampal region, memory performance within each session on the word lists with the temporal cortex stimulation was consistently higher than control lists without stimulation, and above the normal range (FIG. 1 panel C). The same subject also reported subjective experience of improved mental ‘picturing’ of words during the temporal cortex stimulation sessions. Two of the four patients stimulated in the lateral temporal cortex showed a positive effect on memory recall—the other two patients showed a positive trend, which was not observed with stimulation in a different brain region (FIG. 1 panel D). On the level of the whole group, memory recall of the stimulated word lists was significantly higher (paired t-test, p=0.0067, DF=3) than the non-stimulated lists (FIG. 1 panel E). The stimulation had a significant positive effect even in subjects with mild (1050) or no (1111) verbal memory deficits, as described in their respective neuropsychological assessments (Table 1).

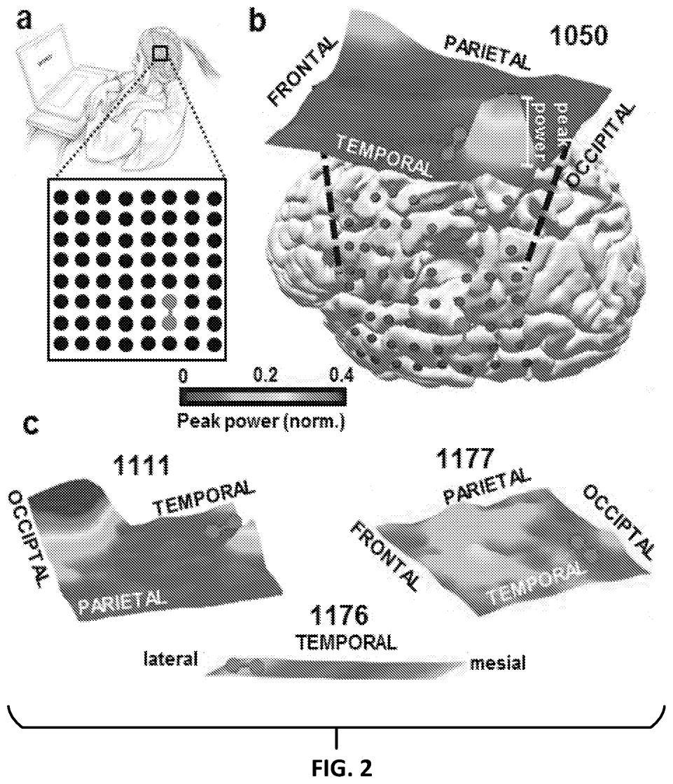

Regarding mapping stimulation sites to electrophysiological activity, each experimental session comprised of 20 lists with stimulation (‘STIM’) and 5 without (‘NON-STIM’; FIG. 1). Stimulation was applied during presentation of two consecutive words, followed by presentation of two other words without any stimulation to enable electrophysiological analysis without stimulus artifact. No difference in recall between stimulated words and the non-stimulated words (paired t-test, p=0.37, N=4, DF=3) on the STIM lists was observed, but the behavioral enhancement was observed on the level of the entire lists. This suggests that the positive effect of stimulation lasted beyond the period of electrical current administration (4.6 s) and modulated encoding of the entire STIM list. To further investigate this behavioral modulation spectral activities were mapped in the electrophysiological recordings induced during encoding of word lists (FIG. 2a, b). High gamma activities (62-118 Hz) were the focus, which have been associated with cognitive processing in humans, and are known to predict successful memory encoding. In this post hoc analysis, the study showed that the stimulation sites in the left lateral temporal cortex were localized in close proximity to discrete foci of induced high gamma response to word presentation in subjects 1050 and 1111 (FIG. 2b, c). The exact location of these high gamma response foci in the temporal cortex were subject-specific and not observed in subjects 1176 and 1177. The high gamma activity foci were not only specific to the language-dominant hemisphere (see Table 1), suggesting activation of a widespread network engaged in these verbal memory tasks. They were not observed in proximity to the stimulation sites in the other three brain areas studied. The four patients were all stimulated in the left lateral temporal cortex that was language dominant (Table 2), although patient 1050 was determined to have bilateral language localization by Wada testing (Table 1).

In order to assess the effect of temporal cortex stimulation on the spectral power, we used power across multiple frequency bands as features for a classifier to further investigate whether the amplitude and frequency parameters could potentially be adjusted for individual patients stimulated in the temporal cortex. To do this the same target electrode was used to test a range of parameters in an additional experiment during quiet wakefulness outside of the task. The fixed parameters that we used in the memory tasks (50 Hz, 1.0-1.5 mA), taken from the previous study (Suthana et al., 2012), were found to be optimal for only one of the four patients (subject 1111) stimulated in the temporal cortex. In two patients of the four patients, higher frequencies (subject 1050) or lower amplitudes (subject 1177) were predicted to exert a greater effect on spectral power modulation and potentially on behavioral performance (not investigated in this study) than the fixed frequency and range of amplitude parameters used to assess the effect on memory encoding in this study. This suggests that stimulation patterns could be optimized to improve the modulatory effect on electrophysiological activity and memory performance.

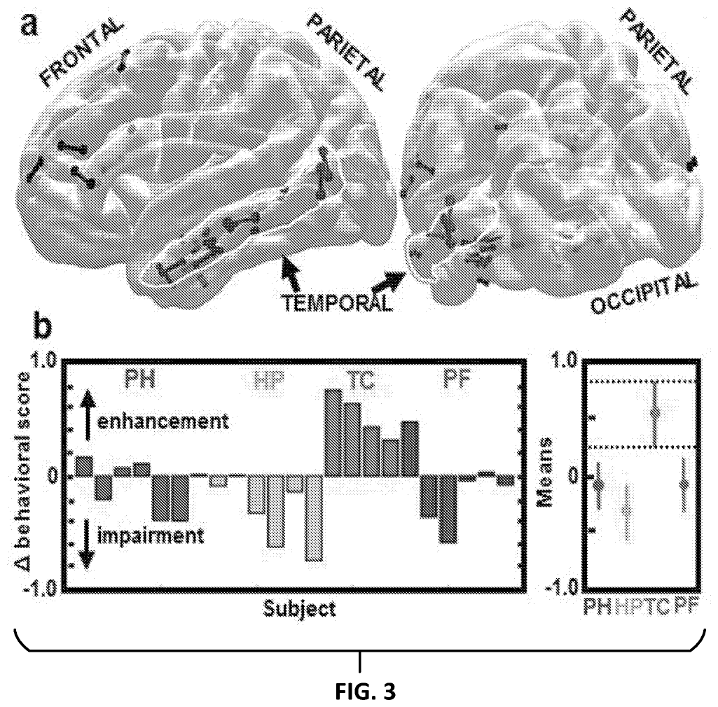

Regarding the effect of stimulation across four regions of the human declarative memory system, the study included testing of whether the behavioral effect of stimulation was specific to the lateral temporal cortex by comparing it to experiments with stimulating electrodes in one of the other three brain regions studied (FIG. 3 panel a). Stimulation had a different effect on memory performance across the brain regions (ANOVA test, p=0.0019, F=7.31, DF=22). The temporal cortex group was different from the other three brain regions stimulated (p<0.05 Tukey post-hoc comparison of 95% CI), showing the only positive effect on memory performance (FIG. 3 panel b). The remaining three groups were not significantly different from each other. The same pattern was confirmed when data from patients, who completed only one session, were included in this analysis, or when data from patients stimulated in the non-dominant hemisphere were excluded—only the temporal cortex stimulation group had a positive effect on verbal memory performance. Probability of obtaining a more positive mean effect using combinations of four randomly drawn scores from all 23 obtained was significantly below chance (permutation test, p=0.0003) even when including the data with patients who completed only one session (p=0.005).

DISCUSSION: The findings show evidence that direct brain stimulation in the dominant lateral temporal cortex can enhance verbal memory in patients. Previous studies, which predominantly stimulated targets in the mesial temporal lobe structures, reported positive and negative effects in other verbal and non-verbal memory tasks (Suthana and Fried, 2014; Kim et al., 2016). Here the study focused on a specific task for verbal short-term memory given evidence from stimulation mapping studies, which suggested involvement of this region in the semantic brain network (Ojemann et al., 1989; Tune and Asaridou, 2016). This region also overlaps with the cortical area mapped with sites where conscious memory experience was elicited in epilepsy patients (Penfield and Perot, 1963). Stimulation sites in this study were localized around the dominant middle temporal gyms, which is associated with processing of semantic information (Binder et al., 2009). Therefore, this brain region presents a viable target for exploring verbal memory enhancement.

The study revealed distinct areas within this region where word encoding induced high gamma activity, which may indicate more precise localization of information processing and thus map potential target sites for stimulation in this and possibly other regions in the temporal cortex. This activity was observed both in the language dominant and non-dominant hemispheres, and beyond the areas mapped during cortical stimulation mapping of language functions performed in a subset of patients. Hence, it is unlikely to be a biomarker of only verbal information processing in these tasks. High frequency activity in the gamma bands and above was previously associated with cognitive processing in human memory tasks in general (Kahana, 2006; Lachaux et al., 2012; Kucewicz et al., 2014) and proposed to reflect the underlying activity of neuronal assemblies. Modulation of this activity with direct electrical stimulation presents one possible mechanism of the reported memory enhancement effect. In the current study, patients that were stimulated in the dominant lateral temporal cortex showed a positive modulation of memory performance.

However, even with direct access of the implanted electrodes to the brain, understanding the electrophysiological effects of the stimulating current propagated over the cortical surface remains a major challenge (Borchers et al., 2012). Hence, it is currently not known whether stimulating in the focus or perimeter of the foci of high gamma activity, on the gyrus or sulcus, from a depth and subdural surface electrode contact, or with different parameters would alter the reported effects. This study as well as other stimulation studies with this patient population are restricted to a limited range of targets and parameters that can be explored, which is dictated by the clinical factors like the areas of epileptogenic or after-discharge activities. Nevertheless, we observed significant memory enhancement in subjects stimulated in proximity of the induced high gamma activity, providing a possible biomarker for the choice of target stimulation sites.

Some aspects regarding the mechanism of the stimulation's effect on electrophysiological activity and memory recall remains to be further explored. For example, it is possible that the temporal cortex stimulation worked by activating a hub of the semantic brain network rather than a single brain region. This hypothesis can be tested in animal models combining other techniques like mapped calcium imaging exemplified in a study of micro-stimulation in rats, which showed a wide-spread activation of sparse assemblies of connected neurons instead of local populations surrounding the stimulating electrode. Using depth or subdural surface electrode contacts is another factor that may influence the modulatory effect of stimulation of neural activities. The spatial scale in either of these two electrode types is unlikely to be optimal for recording, stimulating and modulating neuronal assemblies underlying memory encoding and recall.

Despite these mechanistic limitations, this study advances the field in several important aspects. First of all, this collaborative project overcomes the limit of small number of patients studied in the previous reports of memory enhancement (N<6) from individual research groups, making our larger dataset from multiple sites more reproducible. Secondly, we were able to test the effects of stimulation across four different brain regions. Further, the positive effect of stimulation was reported in individual patients tested across multiple days of stimulation sessions, on the level of the group of patients stimulated in the temporal cortex, and between the four groups stimulated in different brain regions. Previous studies reported the positive effects either as a single case study (Hamani et al., 2008), or as a group effect without a significant enhancement in individual patients (Suthana et al., 2012) or without statistical evaluation (Miller et al., 2015). All of these studies are limited to the number patients available, variable clinical aspects in this patient population like individual case pathologies, medication and cognitive comorbidities, which need to be addressed by further increasing the number of subjects and assessing the effect of baseline deficits in verbal memory functions. Animal model studies are required to address these challenges. Another remaining issue in the field is elucidating the nature of cognitive processes modulated by the stimulation. The stimulation could enhance memory processing per se, or an associated process like attention and perception.

Addressing these and other issues associated with direct brain stimulation for memory enhancement can potentially translate into clinical practice. For instance, the finding that electrical stimulation in the middle dominant temporal gyrus can enhance memory processes might provide a hint as to why some patients undergoing surgical removal of this region complain about verbal memory deficits. Knowledge about patient-specific brain areas involved in verbal memory processing can be used to guide resection surgery or promote alternative stimulation therapies. Additionally, the reported memory enhancement effect may be particularly useful for developing new stimulation treatments for restoring memory functions and thus be applied in the emerging brain-machine interface technologies to treat memory and cognitive functions in humans.

### Pupil Size Reflects Successful Encoding and Recall of Memory in Humans

INTRODUCTION: Pupil size alone is able to predict an overt decision about timing an action and a covert decision about choice of the stimulus, suggesting a link between pupil responses and the higher-order brain systems supporting cognition, decision-making and/or execution of actions.

The anatomy and physiology of the brain pathways controlling pupil size involve both the autonomic and somatic nervous systems. Adrenergic and cholinergic neuromodulation are involved in the regulation of these pathways and, more generally, of the thalamo-cortical brains networks during states of sleep, wakefulness and cognition. The tight link with these wide-spread neuromodulatory systems inspired this research into the relationship between the brain states, electrophysiological activities and the pupil response. Tracking pupil dilation was shown to correlate with transitions in the cortical state as measured in the intracellular membrane potential across multiple brain regions. Furthermore, pupil size and these cortical arousal states were associated with slow and fast electrophysiological activities—low arousal and constricted pupil with low-frequency oscillations, and enhanced sensory responses, arousal and dilated pupil with high frequency oscillations. Hence, pupillometry is an attractive tool for accessing information about the brain states and neurophysiological processes supporting sensory perception, attention and decision-making. Pupil size is modulated not only by the emotional valence and novelty of the presented images, but also by the memory of the familiar ones (‘old/new effect’). Hence, pupillometry provides a signal for ‘strength of memory’, ‘memory retrieval’, and ‘neural novelty’.

One aspect of this study was to determine whether pupil size can be used to predict successful encoding of freely recalled memory. In the recognition memory tasks, pupil responses are compared between either familiar or novel items that are presented for a memory-based decision. It is important to know whether changes in the pupil size during memory encoding can predict subsequent free recall of an item without being presented for choice, and thus alone or accompanied with other modalities of data can provide a biomarker for estimating likelihood of successful memory encoding.

Brain activities measured using electrophysiological and neuroimaging techniques can be used to differentiate stimuli that are likely to be remembered from the ones that will be forgotten. These techniques typically require invasive or expensive recordings of brain activity, and sophisticated tools for data acquisition and analysis. For instance, a recent study applied machine learning approach to predict memory encoding from invasive human recordings during free recall tasks (Ezzyat et al. 2017). A memory signal that can be easily accessed from tracking pupil size and thus by-pass the need for brain recordings would have large impact on the neuroscience research of memory functions and on development of new brain-machine interface technologies to modulate these functions. The biomarker signal could thus be used for e.g. responsive brain stimulation triggered during identified states of low likelihood of memory encoding. Therefore, this study investigated pupil responses across different phases of a free recall memory task in human subjects as they encoded and recalled verbal information.

SUMMARY: This study investigated changes in the pupil size during encoding and recall of word lists. Consistent patterns in the pupil response were found across and within distinct phases of the free recall task. The pupil was most constricted in the initial fixation phase and was gradually more dilated through the subsequent encoding, distractor and recall phases of the task, as the word items were maintained in memory. Within the final recall phase, retrieving memory for individual words was associated with pupil dilation in absence of visual stimulation. Words that were successfully recalled showed significant differences in pupil response during their encoding compared to those that were forgotten—the pupil was more constricted before and more dilated after the onset of word presentation. The results suggest pupil size can be used as a biomarker for probing and modulation of memory processing.

METHODS: Regarding memory task, ten healthy human subjects (five males) of age 20-37 years were recruited to a free recall verbal memory task with eye tracking. First six subjects were tested at the Mayo Clinic in Rochester, Minn., USA, and the last four subjects were tested at the Czech Technical University in Prague, Czech Republic. The task was based on classic paradigms for probing verbal memory, in which subjects learned lists of words for a subsequent recall. Subjects were instructed to study lists of individual words presented sequentially on a laptop computer screen for a later memory test. Lists were composed of twelve words chosen at random from a pool of three hundred high frequency nouns (http://memory.psych.upenn.edu/WordPools). Each word remained on the screen for 1600 ms, followed by a 1000 ms blank interval between stimuli. Immediately following the final word in each list, participants performed a distractor task consisting of a series of arithmetic problems of the form ‘A+B+C=??’, where A, B and C were randomly chosen integers ranging from 1-9. Following the distractor task participants were given 30 seconds to verbally recall as many words as possible from the list in any order. Vocal responses were digitally recorded by the laptop computer and later manually scored for analysis. Each session consisted of seventeen lists of this encoding-distractor-recall procedure.

Regarding tracking of eye movements and pupil dilation, recording of gaze position and pupil size was performed using the ‘i4tracking’ system (Medicton Group Inc.) designed for clinical applications in patients. The recording was performed on a laptop computer connected to a 24-inch monitor screen with resolution of 1680×1050 where the gaze position was tracked by high-resolution (2048×1088) and high-speed (up to 200 Hz) external camera device. Stimuli were displayed on the screen using font size of 100 and were viewed from a distance of approximately 60 cm. Pupil position and size were detected by the camera device, corresponding to approximately 0.1 mm per pixel in the eye image. The camera device was placed below the screen to capture the face area from forehead to the mouth. Two sources of infrared light were emitted from the camera to capture the reflected light for pupil detection. Raw images from the camera were sampled at the rate of 50 Hz and were saved for extracting pupil information using detection algorithms. The algorithms worked by fitting a general ellipse equation over the estimated pupil image. The pupil size in pixels was also converted to millimeters using estimated interpupillary distance (IPD) and the IPD in the camera images. The reported pupil area was computed as an average from both left and right eye using the corresponding vertical and horizontal diameters in ellipse area equation. Gaze position was determined by projecting the movement of the estimated center of the pupil onto the monitor screen area with the use of corneal reflection. Gazes outside of the screen area as well as the eye-blinks were treated as missing-samples. For further analysis, they were filled-in through linear interpolation between the closest samples at each end of the gap to obtain uninterrupted pupil size signal. The total blinking time was determined for each subject and was found to be less than 5% of the total recording time. Vocal responses of the subjects during the recall phase of the task were recorded using a built-in laptop microphone and manually annotated after the experiments in custom software for audio editing.

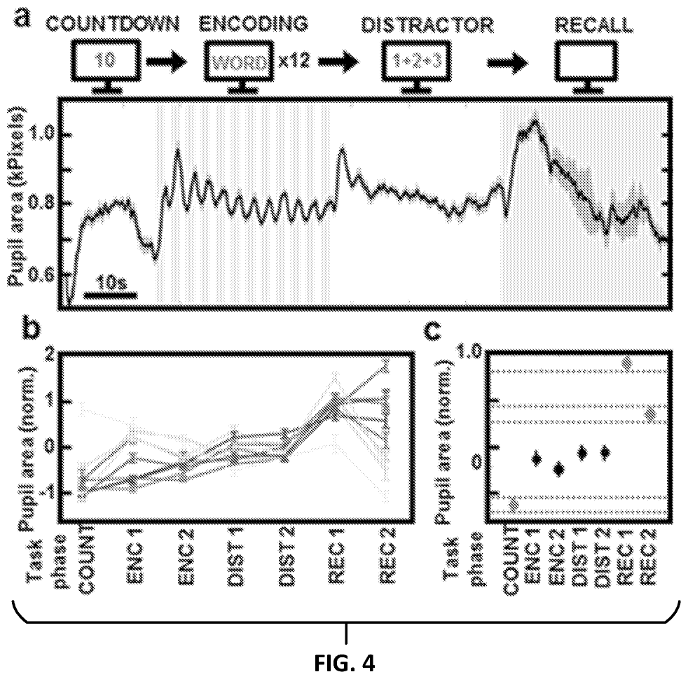

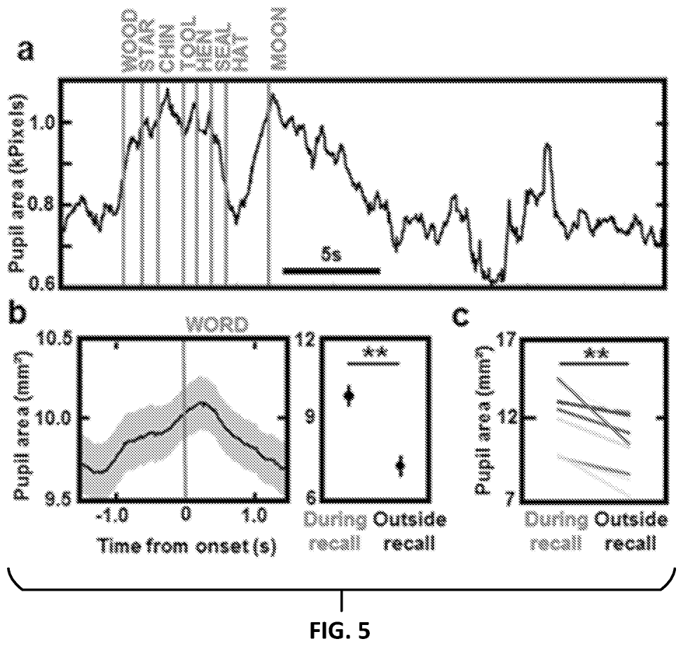

Before presentation of the task word lists the eye tracker was calibrated for each recruited subject. In the calibration procedure, subjects were asked to focus their gaze on nine points presented consecutively at specific positions across the diagonals and centers of the side edges of the display screen. Calibration was repeated throughout the session to ensure accurate estimate of the pupil size. Moreover, subjects were instructed not to move their heads and focus gaze on the screen throughout all phases of the task trials (FIG. 4). This was controlled and quantified by calculating the proportion of time spent gazing outside of a virtual rectangle surrounding the presented word (1.5 times the size of the word—700×200 pixels). All subjects spent negligible amount of time (<5%) blinking or gazing outside of center rectangle during the encoding phase. Only subject 4 spent more than 30% of the time gazing outside of the rectangle area during the recall phase and had to be excluded from the recall phase analysis (FIG. 5). All stimuli were presented on the screen in a light gray color on white background to minimize pupil responses to changing lighting and contrast. The testing was conducted in a room with low-light conditions that remained constant during the testing procedure.

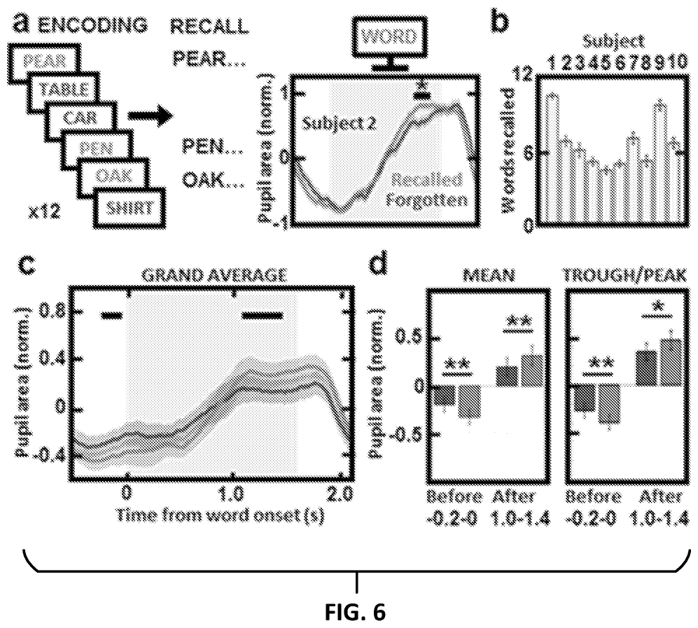

Regarding the analysis of pupil responses, eye blinks were determined by comparing the output of the eye-tracker detection algorithm and three samples preceding and following any missing-value (˜60 ms), which were used to interpolate the estimated pupil size and position during blinking, as described above. Proportion of the gaze focus outside of the screen center, where the stimuli were presented, was computed by dividing the total time outside of the rectangular area centered in the middle of the screen by the total time of uninterrupted eye-tracking without blinking. It was quantified as the raw recording of the pixel area (FIG. 4-6) and also as estimated real area in square millimeters in individual subjects (FIG. 5). For comparisons across different subjects, the raw pupil area was normalized using a z-score transformation by expressing every sample as a standard deviation score from the mean calculated within each word list trial. Average estimates of the normalized pupil size were determined in 12-second time bins of the different phases of the task (FIG. 4) for statistical comparison. Likewise average estimates of the pupil area were determined in the ‘during recall’ epochs surrounding the onset of word vocalization (±1 second before and after the estimated 1-second vocalization time) to compare them to the remaining ‘outside recall’ epochs outside of the vocalization epochs (FIG. 5). Average values of the mean, peak and trough in the pupil response of every subject were determined in two intervals of the encoding phase: ‘before’ and ‘after’ the word presentation from −200 ms to Oms from the onset and from 1000 ms to 1400 ms after the onset, respectively, for comparison between the recalled and forgotten word conditions (FIG. 6).

Regarding statistical analysis, all pupil size data were normalized using the z-score transformation given the approx. normal distribution of the data values in every subject. Two-way ANOVA was used to test the effect of different task phases and subjects on pupil size, which was followed by Tukey-Kramer post-hoc comparison of specific groups (FIG. 4). Paired t-test was used for all the remaining group comparisons of samples taken from the same trial (FIG. 5 panel b, FIG. 6 panel a) or subject (FIG. 5 panel c, FIG. 6 panel d). Bonferroni correction of the p-value was applied for the comparisons of mean and peak/trough values in the two time bins before and after the onset of word presentation (FIG. 6 panel d). All results are presented as mean±S.E.M.

RESULTS: The study employed a classic behavioral paradigm for free recall of verbal information to probe human memory encoding and recall with high-resolution tracking of gaze position and pupil size. The memory task comprised of four successive phases of the encoding-recall procedure (FIG. 4 panel a): ‘countdown’ from 10 to 1 with no memory load, ‘encoding’ of the words displayed individually one after another, ‘distractor’ task completing simple arithmetic equations to prevent rehearsing the word list and minimize the primacy and recency effects, and ‘recall’ when the remembered words were vocalized in any order (see Methods for further details).

Pupil size was remarkably consistent across the entire experimental session and revealed robust changes in the absolute estimate of the area (FIG. 4 panel a). These estimates were normalized for every subject within each encoding-recall procedure of a given word list and averaged in 12 s bins centered in the middle of each phase (encoding, distractor and recall phase were divided into half ‘1’ and ‘2’), showing a trend of increasing pupil size with the successive phases of the task (FIG. 4 panel b). Analysis of variance confirmed a strong effect of the phase (ANOVA, F=195.4, 6 d.f., p<0.0001), no effect of the subject (F=0.47, 9 d.f., p=0.90), and a significant interaction between the phase and subject (F=22.52, 54 d.f., p<0.0001). Pupil size was the largest in the final two recall phases and most constricted in the first countdown phase (FIG. 4 panel c), compared to any other phase of the task (Tukey-Kramer post-hoc comparison, p<0.05). Since there was no memory component in the countdown phase and memory for words was gradually added and maintained along successive phases of the task, this general pattern suggests a correlation between cognitive load in the task and pupil dilation as previously proposed.

Regarding pupil size during free recall of memory, assuming that pupil dilation correlates with cognitive load or effort in the task, it would be expected to be different at times when words are being recalled from memory and when they are not. This study revealed that large pupil dilation at times when subjects were actively recalling words (FIG. 5 panel a), which could not be attributed to any changes in the screen display (screen was blank during the entire recall phase) or lighting in the room. This increase in the pupil size started rising before the onset of the vocal response and gradually decreased afterwards (FIG. 5 panel b), which does not exclude a possibility that the two may be related through a preparatory process initiated before the response. Recall epochs around this response (ls before and after word vocalization) were characterized by greater pupil size as compared to the recall epochs outside of these vocalizations (FIG. 5 panel b). This effect was significant in individual subjects (FIG. 5 panel b) and on the group level (paired t-test, N=9, p=0.0024; one subject was excluded from this analysis based on proportion of time during recall with eye-tracking outside of the screen area—see Methods) with each individual subject showing a greater mean of the absolute pupil size in the word recall condition (FIG. 5 panel c). Therefore, pupil size reflected a cognitive process associated with active recall of the encoded memory on the level of individual subjects and the whole group.

Regarding pupil response to encoding of remembered and forgotten words, to further investigate possible cognitive processes reflected by the pupil response, the study compared the encoding periods of words that were subsequently remembered and recalled to those that were not. Pupil responses were normalized for each word list by transforming the raw signal during the encoding phase into z-scores (see Methods). The normalized responses were then compared between the recalled and forgotten word conditions (FIG. 6 panel a). Subjects showed a consistent pattern of response to word encoding—initial pupil constriction was followed by dilation peaking toward the end of word presentation on the screen, at which point the greatest difference between the two conditions was observed (FIG. 6 panel a). Despite considerable variability in pupil response patterns and subject memory performance ranging from five to ten words recalled on average (FIG. 6 panel b), the pupil response pattern revealed consistent trends across different subjects. The greatest difference between the two conditions was in the first 200 ms ‘before’ and 1000 ms ‘after’ the word onset with more constricted and dilated pupil in the recalled word condition, respectively (FIG. 6 panel c). This subsequent memory effect was quantified by comparing mean values in these epochs as well as the peak and through for all subjects (FIG. 6 panel d). Pupil size during encoding of subsequently recalled words had significantly lower mean (paired t-test, N=10, p=0.0044) and through (p=0.0015) values before word onset, and significantly higher mean (p=0.0021) and peak (p=0.0121) values after the onset. The findings show that pupil reaction right before and during presentation of the stimuli can be used to predict their subsequent memory recall.

DISCUSSION: The results show that the signal sampled from tracking changes in pupil area contains information about the brain states and cognitive processes underlying memory encoding, maintenance and recall. A general pupil size increase with mental effort and difficulty across the successive phases of the task was observed. Task difficulty was increased from the encoding through the distractor phase of the task as the memory for words had to be maintained and freely recalled during the final phase when the pupil size was at its largest. There was a significant drop in the pupil size going from the first to the second half of the recall phase (FIG. 4), which can be explained by gradual ‘unloading’ of the actively maintained items from a memory buffer. Most of the words were recalled in the first half of this phase. Recalling a word was associated with ramping up of the pupil size, which started before the time of vocalization (FIG. 5), which may be related to preparatory perceptual, cognitive or motor processing. In the encoding phase, pupil size was also consistently ramped up after presentation onset peaking at longer latencies above 800 ms (FIG. 6) when one would expect subject engagement in creating mental representations (e.g. visual depiction or words), active rehearsal, or other strategies employed for enhanced memorization. Both the gradual ‘macro-scale’ increase across the task phases and the ‘micro-scale’ pupil dilation around the recall and encoding of individual words suggest pupil size as an indicator of the processes engaged in storing, maintaining and retrieving information.

For an indicator of brain processes involved in these complex cognitive functions, pupil responses were found to be remarkably robust across subjects. Pupil responses varied between different subjects, showing patterns specific to a given individual. In these subject-specific differences, consistent changes in the pupil response both on the level of the task phases and presentations of individual words for encoding were observed. The latter showed an initial constriction of the pupil size before the presentation followed by a later dilation during and beyond the interval of word display on the screen (FIG. 6). On the level of individual subjects, the mean and trough of the constriction, and the mean and peak of the dilation were different between words that were subsequently recalled and those that were not at very specific times of word encoding. Although such subsequent memory effect was reported in electrophysiological and brain imaging studies, it would not be expected to be as consistent in its latency and across different subjects. The BOLD signal is limited in its temporal resolution, whereas the electrophysiological signals show variable latencies depending on the brain region and the frequency band analyzed. In the same tasks, power changes in the gamma frequency bands revealed a similar pattern of decreased activity before word presentation and increased activity afterwards on the trails with subsequently recalled words. Latency and magnitude of this electrophysiological subsequent memory effect was more variable than the pupil size responses and less generalizable. This study was also limited to a low number of subjects tested relative to the studies comparing brain activity. In spite of a low number of subjects and trials, and the individual differences in the tested group of subjects, there were still significant differences at the time of the trough and peak of the pupil response. Similar differences were observed with the gamma activity, which altogether could reflect decreased encoding in preparation for word onset (pupil constriction and decreased gamma) followed by enhanced encoding during the presentation time (pupil dilation and enhanced gamma). High-resolution tracking of the pupil size, therefore, provides a new biomarker for memory processing, complementary to the currently used brain activity measures and advantageous in terms of its accessibility and robustness.

Moreover, pupil dilation and the electrophysiological measures of memory processing recordings focused on tracking the gaze were done in studies with non-human primates. Phase reset in low-frequency oscillations and increased incidence of high frequency oscillations, called the sharp-wave ripples, were associated with memory performance and eye movements to remembered stimuli. Elucidating the relationship between the eye-tracking and electrophysiological measures assists the understanding of these biomarkers and the brain mechanisms supporting memory processing. Eye-tracking can help to dissociate brain activities underlying memory processing from perception, attention and decision-making by following saccades, fixations and pupil dilation. Furthermore, specific brain activities can be correlated with specific eye-tracking features. For example, recent rodent studies correlated sharp-wave ripples in the hippocampus with pupil dilation and brain states of arousal and attention. Similarly, sharp-wave ripples in primates were reported in response to the stimuli that were attended to with smaller saccades and longer fixations, which increased the probability of perceptual detection. In another study, the sharp wave ripples occurring around the time of fixations on stimuli were shown to be indicative of their subsequent memory. Human studies employing new techniques for recording these high frequency activities together with advanced high-resolution eye-tracking will shed more light on the underlying neuronal processes.

This study infers aspects about memory processes from the behavioral measure of pupil size responses in a free recall task. The study observed pupil responses in the absence of visual stimulation during recall and no consistent responses to the countdown numbers presented on the screen. Therefore, these pupil responses were not driven by visual stimulation, suggesting that other sensory modalities of the presented stimuli, e.g. auditory tones, could induce similar responses. Modality-independence can be particularly important for applying pupil responses in memory enhancement technologies to trigger modulation of brain activity. For instance, pupil size can provide a non-invasive biomarker for brain stimulation during predicted states of poor memory encoding. Using pupil dilation to trigger brain stimulation would also provide a direct test of the relationship with memory processing and the underlying brain activity. Knowledge from combined recordings of brain activity and eye responses can be directly implemented into the emerging neuromodulation technologies.

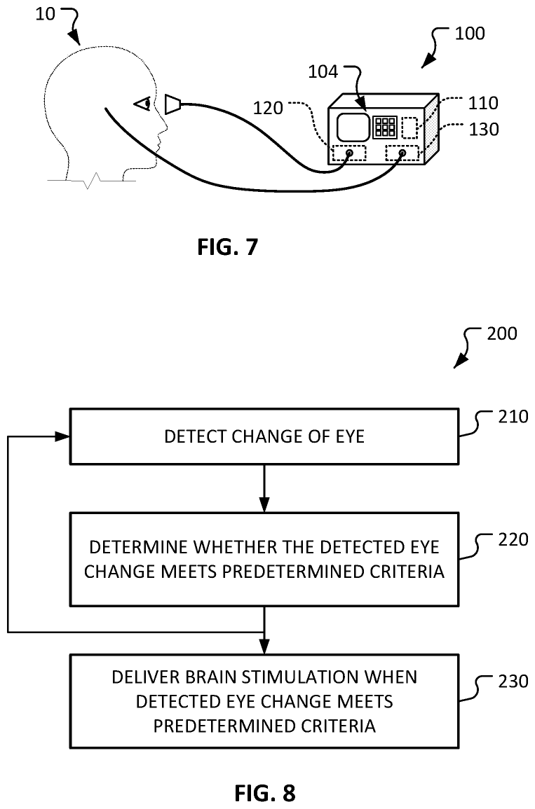

Referring to FIG. 7, an example system 100 can be used to enhance the memory and/or cognitive performance of a patient 10. The system 100 monitors the eye(s) of the patient 10 for changes, and delivers electrical stimulation to the lateral temporal cortex of the patient's brain when changes in the eye(s) are detected that meet or exceed pre-determined criteria.

In some embodiments, the system 100 is capable of on-going adaptive training to select optimal parameters for brain stimulation in an individual patient 10. This can be achieved, for example, through memory task performance on a hand-held device, which is wirelessly connected to cloud-computing to upload data from memory performance, gaze tracking, and pupillometry, or pupillometry with/without intracranial electrophysiology or other modalities of the data. As a result, memory performance can be improved in daily lives. Current brain stimulation technologies do not use eye-tracking signals to control and train the stimulation patterns in a closed-loop. None of the current brain stimulation technologies use personalized training of algorithms controlling the stimulation. Most of the existing systems employ open-loop stimulation with set options of parameters and algorithms designed for a general population. This disclosure includes a paradigm for memory tasks with stimulation, which can be applied with the lateral temporal cortex as the target or in a closed-loop with combined tracking of gaze position and pupillometry. In some embodiments, the system 100 is configured as a hand-held device with wireless connection to cloud computing.

The system 100 can include a controller 110, an eye-change detection sub-system 120, and an electrical brain stimulation sub-system with both stimulation and recording capability in 130. The eye-change detection sub-system 120 and the electrical brain stimulation sub-system 130 are each in signal communication with the controller 110 and are responsive thereto.

The controller 110 can include, for example, a combination of processor(s) and computer-readable memory (which may store executable instructions configured to perform the operations of method 200 described by FIG. 8). The processor(s) can be suitable for the execution of one or more computer programs and can include, by way of example, both general and special purpose microprocessors, and any one or more processors of any kind of digital computer. The controller 110 may be implemented as a chipset of chips that include separate and multiple analog and digital processors. Such processor(s) may provide, for example, for coordination of the other components of the system 100, such as the eye-change detection sub-system 120 and an electrical brain stimulation sub-system 130, applications run by the system 100, a user interface 104 of the system 100, and communications with other systems/devices.

To provide for interactions with a user, the system 100 can also include a user interface 104. The user interface 104 includes devices and systems to receive inputs to the system 100, and to provide outputs from the system 100. For example, in some embodiments the user interface 104 can include a display (in some embodiments the display is a touchscreen display), one or more buttons that can be soft keys or hard keys, one or more audio speakers, one or more lights, a microphone, a camera, tactile feedback mechanisms (e.g., vibratory alarm signals), and the like. Using such devices, the user interface 104 can receive user input including voice input, touchscreen input, soft key inputs, and the like. The user interface 104 can also provide outputs including audible alarms or messages, visual alarms or messages, tactile alarms or messages, differentiation of alarm types, and the like.

The system 100 includes the eye-change detection sub-system 120, which is a sub-system for visually monitoring at least one eye of the patient 10. For example, in some embodiments, a camera system is used to monitor the eye(s) of the patient (e.g., to track eye movements and pupil dynamics). In some embodiments, the eye-change detection sub-system 120 includes visual recognition functionality. Accordingly, the eye-change detection sub-system 120 can serve to monitor at least one eye of the patient 10 and, in conjunction with the controller 110, changes thereof. For example, in some embodiments the eye-change detection sub-system 120 (and optionally in conjunction with the controller 110) can monitor and/or detect changes in at least one eye of the patient 10 such as pupil dilation (e.g., pupillometry), pupil constriction, pupil dynamics, eye movement, gaze-tracking and the like, and combinations thereof. Measurements of pupil dilation and eye movement alone or together with other modalities of the data are used to tune stimulation to place the brain in an optimal state for cognitive and memory performance.

The system 100 also includes the electrical brain stimulation sub-system 130. The electrical brain stimulation sub-system 130 is activated and otherwise controlled by the controller 110 of the system 100. The electrical brain stimulation sub-system 130 can include one or more leads and/or electrode probes that can be utilized to deliver an electrical stimulation to the brain of the patient 10, or also record electrophysiological signals or other modalities of the data that may also feed system 100 via controller 110. For example, in some cases an electrical stimulation can be delivered from the electrical brain stimulation sub-system 130 to a particular location of the patient's brain such as, but not limited to, the lateral temporal cortex of the brain of the patient 10 based on inputs from the same electrodes being utilized for stimulation or via control input from sub-system 120.

Referring to FIG. 8, a method 200 can be used to enhance the memory and cognitive performance of a patient. In some embodiments, the method 200 can be implemented using the system 100 of FIG. 7.

At step 210, a change in an eye of a patient is detected. Such changes can include, but are not limited to, pupil dilation (e.g., pupillometry), pupil constriction, eye movement, gaze-tracking, and the like, and combinations thereof. Measurements of pupil dilation and eye movement alone or together with other modalities of the data are used to tune stimulation to place the brain in an optimal state for cognitive and memory performance.

At step 220, the eye change(s) detected in step 210 is/are assessed to determine whether the change(s) meets or exceeds predetermined criteria. For example, in the context of system 100 of FIG. 7, the controller 110 can receive one or more signals from the eye-change detection sub-system 120 and then compare and/or synthesize the one or more signals in accordance with an algorithm to determine whether the change meets or exceeds predetermined criteria (which may be individualized criteria in some cases) that are stored and or programmed in the controller 110.

At step 230, and in response to a determination that the detected eye change meets or exceeds the predetermined criteria from step 220, electrical brain stimulation can be delivered to a patient. For example, in the context of system 100 of FIG. 7, the electrical brain stimulation sub-system 130 can be activated by the controller 110 to deliver electrical brain stimulation to the patient. In some cases, an electrical stimulation can be delivered from the electrical brain stimulation sub-system 130 to a particular location of the patient's brain such as, but not limited to, the lateral temporal cortex of the brain of the patient 10. Such an electrical stimulation to the brain that is delivered in response to the detection of a change in the patient's eye (e.g., pupil dilation or other types of changes/movements meeting/exceeding predetermined criteria) has been found to be effective for enhancing the memory and/or cognitive performance of a patient 10.

While this specification contains many specific implementation details, these should not be construed as limitations on the scope of any invention or of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described herein as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described herein should not be understood as requiring such separation in all embodiments, and it should be understood that the described program components and systems can generally be integrated together in a single product or packaged into multiple products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In certain implementations, multitasking and parallel processing may be advantageous.

1. A system for cognitive performance or memory enhancement therapy system, comprising:
a controller;
an eye-change detection sub-system in signal communication with the controller; and
an electrical brain stimulation sub-system in signal communication with the controller.

2. The system of claim 1, wherein the eye-change detection sub-system comprises one or more cameras.

3. The system of claim 1, wherein the controller is configured for adaptive training.

4. The system of claim 1, wherein the system is a hand-held device.

5. The system of claim 1, wherein the eye-change detection sub-system is configured to detect changes to pupil size and/or gaze position.

6. The system of claim 1, wherein the system includes an eye-change recording means.

7. The system of claim 1, wherein the electrical brain stimulation sub-system includes one or more leads and/or electrode probes that can deliver electrical stimulation to a brain of a patient and/or record electrophysiological signals or other modalities from the brain of the patient.

8. A method for enhancing memory or cognitive performance of a patient, comprising:
detecting a change in an eye of the patient;
comparing the change to predetermined criteria; and
in response to the change meeting the predetermined criteria, delivering electrical brain stimulation.

9. The method of claim 8, wherein the change comprises a dilation or constriction of a pupil.

10. The method of claim 8, wherein the change comprises an eye movement or a change in a gaze of the eye of the patient.

11. The method of claim 8, wherein the electrical brain stimulation is delivered to a lateral temporal cortex of the patient.

12. A method for enhancing memory or cognitive performance of a patient, comprising:
detecting a change in an eye of the patient;
correlating the detected change in the eye of the patient with electrophysiologic signals from within a brain of the patient; and
in response to the correlation meeting predetermined criteria, delivering electrical brain stimulation.

13. The method of claim 12, wherein the electrical brain stimulation is delivered to a lateral temporal cortex of the patient.
